(19) United States
(12) Patent Application Publication
(10) Pub. No.: US 2016/0082087 A1
(43) Pub. Date: Mar. 24, 2016

(54) WHITENING PROMOTING AGENT OR ATOPIC DERMATITIS AMELIORATING AGENT

(71) Applicant: NITTA GELATIN INC., Osaka (JP)

(72) Inventors: Seiko KOIZUMI, Osaka (JP); Fumihito SUGIHARA, Osaka (JP); Naoki INOUE, Osaka (JP)

(21) Appl. No.: 14/786,378

(22) PCT Filed: Mar. 31, 2014
(86) PCT No.: PCT/JP2014/059408

§ 371 Date: Oct. 22, 2015

(30) Foreign Application Priority Data
Apr. 26, 2013 (JP) 2013-093591
Oct. 31, 2013 (JP) 2013-226312

(51) Int. Cl. A61K 38/39; A61Q 19/02; A61K 8/64

(52) U.S. Cl. CPC A61K 38/39; A61K 8/64; A61Q 19/02

(57) ABSTRACT

A peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof is useful as a whitening promoting agent or atopic dermatitis ameliorating agent.

### TECHNICAL FIELD

The present invention relates to a whitening promoting agent or atopic dermatitis ameliorating agent comprising a peptide or the like.

### BACKGROUND ART

Ultraviolet radiation is considered as a cause of maculae and freckles of skin. The process of formation of pigmentation due to ultraviolet radiation is roughly divided into three stages: proliferation of melanocytes, synthesis and activation of tyrosinase which is a rate-determining enzyme of melanization, and transportation of melanosomes from melanocytes to keratinocytes. In particular, cytokines that act on melanocytes paracrinally from epidermal keratinocytes after UVB irradiation are greatly involved in the first two stages, and endothelin-1 (ET-1), basic fibroblast growth factor (bFGF), α-melanocyte stimulating hormone (aMSH), membrane-bound stem cell growth factor (SCF), nitrogen monoxide (NO) and so on have been reported heretofore. Therefore, pigmentation can be suppressed by suppressing these cytokines, for example, by suppressing expression of endoserine-1 (NPD 1).

Skin is composed of an epidermis layer and a dermis layer, and it has been revealed that in addition to epidermis layer cells, conifered envelope (CE) which is a marginal structure also has an important role in the barrier function. As an epidermal keratinocyte differentiates, precursor proteins that form CE emerge in the site spanning from a stratum spinosum upper layer to a granular layer. Then, in the course of reaching a horny layer, an isopeptide bond is formed between a lysine residue and a glutamine residue of these proteins, and thus cross-linking and insolubilization occur to complete CE. Existence of a pseudo isopeptide bond formed between glutamine residues via polyamine is also known. Formation of these bonds is catalyzed by transglutaminase (TGase) which is an enzyme produced in association with differentiation of an epidermal keratinocyte. TGase includes several isozymes, and among these, TGase3 existing in cytoplasm and membrane-bound TGase1 is presumed to have an important role for formation of CE. Transglutaminase is responsible for cross-linking of structural proteins, but does not have a function of adhering proteins in the stage that the epidermis is still undifferentiated in a deep space. Then, there is an ingenious mechanism that as the epidermis is matured and differentiated, the precursors having low activity gradually get to have activity and start functioning. Examples of precursor proteins constituting CE include involucrin, loricrin, small proline rich protein (SPR, cornifin), cystatin A, elafin, filaggrin, keratin, envoplakin, desmosome constituting protein, squireline, annexin 1, and PAI-2. As these precursor proteins increase, CE also increases, and thus the barrier function improves. Filaggrin is one of the basic proteins produced in an epidermal granular cell, and has a role of suppressing immune response against a foreign matter by forming a horny layer that is essential for the function of ameliorating atopic dermatitis to thereby improve the barrier function. It is biosynthesized as a precursor profilaggrin, and in the stage that a horny layer is formed, profilaggrin phosphate undergoes dephosphorylation and limited hydrolysis to form filaggrin. Further, filaggrin is degraded to form a natural moisturizing factor (NMF). Dryness is also a factor of making atopic dermatitis worse. It is conceivable that increase in filaggrin which is a source of an NMF and promotion of epidermal hyaluronic acid production also improve the moisture retention and ameliorate the dryness of the skin likewise an NMF, and thereby ameliorate atopic dermatitis.

Therefore, expression of transglutaminase 1 (TGM1), involucrin (ivl), keratin 10 (K10) and filaggrin (FLG) is an indicator of the final stage of the differentiation process. By promoting expression of transglutaminase 1, involucrin, keratin 10 and filaggrin, epidermal metabolism, namely metabolism of epidermal keratinized cells is promoted, so that the epidermal keratinized cells will not be held in the skin surface as maculae or freckles, and discharge of melanin granules from the skin can be promoted. Further, since atopic dermatitis is associated with reduction in filaggrin protein and deterioration in moisture retention, enhancement of expression of a filaggrin gene and enhancement of expression of a hyaluronic acid synthase 2 gene contribute to amelioration of atopic dermatitis.

NPD 2 describes that when collagen was orally administered to a pseudo-aged mouse whose collagen synthesis was suppressed to 40% of that of a normal mouse, the collagen synthesis amount was restored to 98%, and the skin turnover was promoted by about 20%. According to the literature, this experimental result is attributed to promotion of collagen synthesis of the dermis layer supporting the basal layer of the skin, and activation of metabolism of the epidermis in cooperation with this. However, NPD 2 lacks description about a collagen peptide mixture and so on, and does not show the effect of promoting whitening because the experiment uses a pseudo-aged mouse in which collagen synthesis is abnormally reduced.

NPD 3 describes that a collagen peptide mixture derived from jelly fish has an antioxidative effect, and reduced the amount of melanin. Since the experimental system of NPD 3 is different from the experimental system of the present description, and comparison of the effect with the present invention cannot be made, we prepared the collagen peptide mixture of NPD 3 as in the later-described Comparative Example 24, and made an evaluation test. As a result, it was revealed that the effect was markedly inferior to that of the present invention.

NPD 4 describes that deerhorn glue has a filaggrin expression promoting ability. However, as is apparent from the later-described Comparative Example 26, the evaluation test conducted by using purchased deerhorn glue revealed that the effect was markedly inferior to that of the present invention.

NPD 5 reports that a collagen peptide derived from salmon skin ameliorates atopic dermatitis by the back of the neck score. However, an evaluation test conducted by using a purchased marine collagen peptide in the manner as described in the later-described Comparative Example 27 revealed that the effect was markedly inferior to that of the present invention.

PTD 1 describes an external preparation for ameliorating cutaneous pruritus caused by atopic dermatitis by a tripeptide. The peptides in PTD 1 are different in sequence from the peptides of the present invention, and amelioration of cutaneous pruritus caused by atopic dermatitis, which is an intended use thereof, differs from amelioration of the essence of atopic dermatitis itself intended by the present invention. PTD 2 reports that no significant difference was observed in all measured items including cutaneous pruritus caused by atopic dermatitis, measurement of total IgE amount in blood, transepidermal water loss (TEWL), and acidophil count and mast cell count in the skin when the one similar to the collagen peptide in PTD 1 was used. Also we purchased these collagen peptides and simultaneously compared these as described in the later-described Comparative Example 25, and confirmed that the effect was markedly inferior to that of the present invention.

### CITATION LIST

### Patent Document

PTD 1: Japanese Patent Laying-Open No. 2003-137807
PTD 2: WO2011/070767

### Non Patent Document

NPD 1: J. Invest. Dermatology, 105: 32-37 (1995)
NPD 2: Fragrance Journal, 1997-7: 58-64
NPD 3: J. Sci. Food Agric., 89: 1722-1727 (2009)
NPD 4: Int. J. Cosmet. Sci., 35(3): 281-285 (2013)
NPD 5: Northern Advancement Center for Science & Technology, Reports of Research & Development subsidizing projects 2005 (approved in 2004), pp. 161-174

### SUMMARY OF INVENTION

### Technical Problem

An object to be achieved by the present invention is to provide an excellent whitening promoting agent or atopic dermatitis ameliorating agent.

### Solution to Problem

Through diligent efforts, the present inventors found that a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp has an excellent endoserine-1 expression suppressing effect, and also has an effect of promoting expression of transglutaminase 1, involucrin, keratin 10, filaggrin and hyaluronic acid synthase 2, and finally accomplished the present invention. Specifically, the present invention is as follows.

[1]A whitening promoting agent or atopic dermatitis ameliorating agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.

[2] The whitening promoting agent or atopic dermatitis ameliorating agent according to [1], comprising a collagen peptide mixture comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof so that the total content of the peptide or the pharmaceutically acceptable salt thereof is 1.6% by weight or more.

[3] The whitening promoting agent or atopic dermatitis ameliorating agent according to [1], comprising a collagen peptide mixture comprising a peptide selected from the group consisting of Hyp-Gly, Pro-Ala-Gly, Gly-Pro, Glu-Hyp-Gly, Glu-Hyp, Ala-Hyp-Gly, Ser-Hyp-Gly and Phe-Hyp or a pharmaceutically acceptable salt thereof so that the total content of the peptide or the pharmaceutically acceptable salt thereof is 1.6% by weight or more.

[4] The whitening promoting agent or atopic dermatitis ameliorating agent according to any one of [1] to [3], which is administered orally or percutaneously.

[5] An endoserine-1 expression suppressing agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.

[6]A filaggrin expression promoting agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.

[7]A method for promoting whitening or method for ameliorating atopic dermatitis, comprising applying a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof to a subject in need thereof.

[8]A method for suppressing expression of endoserine-1 or method for promoting expression of filaggrin, comprising applying a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof to a subject in need thereof.

### Advantageous Effects of Invention

A peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp suppresses expression of endoserine-1, and thus suppresses generation of melanin pigments from melanocytes, and suppresses pigmentation. Further, these peptides promote expression of transglutaminase 1, involucrin, keratin 10 and filaggrin, and thus promote epidermal metabolism to promote turnover of the skin, and make melanin pigments (maculae) in the skin to be discharged more quickly. These two effects promote whitening.

Further, a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp improves the skin barrier function by promoting expression of a filaggrin gene. Further, existence of a natural moisturizing factor (NMF) as a result of degradation of generated filaggrin ameliorates dryness of the skin. Also by promoting production of hyaluronic acid in keratinocytes, it is possible to improve the moisture retention of epidermis and ameliorate dryness of the skin as is the case with an NMF. While depression in internal immunity is one of the causes of atopic dermatitis, an internal IgE value was significantly suppressed, and depression in allergic immunity was observed when a collagen peptide mixture comprising these peptides at high concentration was ingested. These two effects ameliorate atopic dermatitis.

### DESCRIPTION OF EMBODIMENTS

Hereinafter, the present invention will be described in detail.

### 1. Peptide

Peptides used in the present invention are Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp (hereinafter, also referred to as the present peptides), and the present peptide may be a pharmaceutically acceptable salt thereof. Preferred peptides include Hyp-Gly, Pro-Ala-Gly, Gly-Pro, Glu-Hyp-Gly, (Pro-Hyp-Gly)₅ and (Pro-Hyp-Gly)₂, and more preferred peptides include Hyp-Gly and Pro-Ala-Gly. The present peptide may be a combination of two or more peptides. Also a collagen peptide mixture comprising these peptides or a pharmaceutically acceptable salt thereof can be used. In this case, the total content of these peptides or a pharmaceutically acceptable salt thereof are 1.6% by weight or more, preferably 1.8% by weight or more, more preferably 2.0% by weight or more, further preferably 2.3% by weight or more, and particularly preferably 2.6% by weight or more. Also preferred is a collagen peptide mixture comprising a peptide selected from the group consisting of Hyp-Gly, Pro-Ala-Gly, Gly-Pro, Glu-Hyp-Gly, Glu-Hyp, Ala-Hyp-Gly, Ser-Hyp-Gly and Phe-Hyp or a pharmaceutically acceptable salt thereof so that the total content of these peptides or a pharmaceutically acceptable salt thereof is 1.6% by weight or more, preferably 1.8% by weight or more, more preferably 2.0% by weight or more, further preferably 2.3% by weight or more, and particularly preferably 2.6% by weight or more. Preferred examples include, for example, a collagen peptide mixture “Type-S” (available from Nitta Gelatin Inc.) comprising 2.94% by weight of these peptides.

Examples of “pharmaceutically acceptable salt” include inorganic acid salts such as hydrochlorides, sulfates, phosphates, and hydrobromides, organic acid salts such as acetates, methanesulfonates, benzenesulfonates, p-toluenesulfonates, succinates, oxalates, fumarates, and maleates, inorganic basic salts such as sodium salts, potassium salts, and calcium salts, and organic basic salts such as triethyl ammonium salts. According to a usual method, a specific peptide can be made into a pharmaceutically acceptable salt thereof.

The present peptide can be synthesized from amino acids by using, for example, a solid phase synthesis method or a liquid phase synthesis method (for example, Japanese Patent Laying-Open No. 2003-183298). In the case of a solid phase synthesis method, an Fmoc method and a Boc method are known, and the present peptide can be synthesized by either method. An example of a solid phase synthesis method will be concretely described below. A bead of polystyrene polymer gel having a diameter of about 0.1 mm that is modified on its surface with an amino group is used as a solid phase, and diisopropylcarbodiimide is used as a condensing agent. First, an amino group of an amino acid at the C terminal is protected with an Fmoc group or a Boc group, and allowed to form a peptide bond with an amino group of the above-mentioned polystyrene polymer gel. The solid phase is washed well with a solvent, and the remaining reagent and amino acid are removed by washing, and then the protecting group of the amino group of the amino acid bound to the solid phase is removed. Then, by sequentially repeating the reaction as described above by using an amino acid whose amino group is protected, a peptide is synthesized on the solid phase. Lastly, the solid phase is digested with trifluoroacetic acid to separate the peptide from the solid phase, and thus a peptide can be synthesized.

The present peptide can also be produced by hydrolyzing gelatin in combination with two or more kinds of endo-type protease and exo-type protease. In the present invention, the above-mentioned hydrolyzed collagen peptide mixture or a mixture obtained by partially purifying the same can also be used. The hydrolysis in combination of two or more kinds, and the purification can be conducted, for example, by referring to the method described in WO2012/081531, WO2012/102308 and so on.

In the present invention, the present peptide may be chemically modified. Chemical modification can be conducted for each amino acid, for example, a hydroxyl group of hydroxyproline, an amino group of an N-terminal amino acid, and a carboxyl group of a C-terminal amino acid. Such chemical modification allows dissolution under weakly acidic to neutral conditions, and makes it possible to improve the compatibility with the later-described other active ingredients.

Concretely, chemical modification of a hydroxyl group of hydroxyproline includes, for example, O-acetylation. Chemical modification of an amino group of an N-terminal amino acid includes, for example, polypeptidylation, succinylation, maleylation, acetylation, deamination, benzoylation, alkylsulfonylation, allylsulfonylation, dinitrophenylation, trinitrophenylation, carbamylation, phenylcarbamylation, and thiolation. Chemical modification of a carboxyl group of a C-terminal amino acid includes, for example, esterification and amidation. Further, in the case of cationizing the present peptide, ethylenediamination, spermination and the like can be conducted.

As to concrete means and treatment conditions of chemical modification, ordinary chemical modification techniques for peptides are applied. For example, O-acetylation of a hydroxyl group of hydroxyproline can be conducted by reacting with acetic anhydride in an aqueous solvent or a nonaqueous solvent. For example, esterification of a carboxyl group of a C-terminal amino acid can be conducted, for example, by aerating a suspension in methanol with dry hydrogen chloride gas, and amidation thereof can be conducted by reacting with carbodiimide or the like. Further, as other concrete examples of chemical modification, chemical modification techniques described in Japanese Patent Publication No. 62-44522 and Japanese Patent Publication No. 5-79046 can be applied.

### 2. Whitening Promoting Agent or Atopic Dermatitis Ameliorating Agent

As will be described in the later-described test examples, the present peptide has an endoserine-1 expression suppressing effect. It also has an effect of promoting expression of epidermal transglutaminase 1, involucrin, keratin 10, filaggrin and hyaluronic acid synthase 2. These two effects suppress generation of melanin pigments, and cause the melanin pigments in the skin to be discharged more quickly, and hence provide the use as a whitening promoting agent or atopic dermatitis ameliorating agent. Concrete applications include pharmaceuticals, cosmetics, foods for specified health use, health foods, and administration to subjects such as human beings and mammals in the state of being contained in various food materials.

The whitening promoting agent or atopic dermatitis ameliorating agent of the present invention can be administered in various forms orally or parenterally. Examples of forms for oral administration include tablets, granules, capsules, powders, liquids, suspensions, and emulsions, and mixtures in beverages or foods. Examples of forms for parenteral administration include application to skin, injections, percutaneous agents, suppositories, nasal drops and inhalants. Preferred examples include tablets, granules, capsules, liquids for direct application to skin, films, ointments, creams, and cataplasms. The present peptide is suited for ingestion by oral administration because it is little digested into amino acids in the digestive tract, and rapidly absorbed in the intestinal tract. It is also preferred that the present peptide is ingested in the state of being mixed in a meal or beverage.

The dose of the present peptide depends on the condition and body weight of the subject, kind of the compound, an administration route and so on, and is, for example, about 0.1 to 2000 mg, preferably about 1 to 1000 mg, more preferably about 5 to 500 mg, particularly preferably about 10 to 200 mg per day per one adult person in the case of oral administration. When the peptide is directly administered to the skin, the content of the present peptide in the entire percutaneous agent is, for example, about 0.00001 to 20% by weight, preferably about 0.0001 to 10% by weight, more preferably about 0.001 to 5% by weight. Regarding preparations of other forms, the dose can be appropriately determined by reference to these doses. These preparations can be administered once or several times in a day, or administered once every one to several days. When the preparation is used in the form of a collagen peptide mixture, adjustment can be made so that the present peptide contained therein has the dose or the content as described above.

The whitening promoting agent or atopic dermatitis ameliorating agent of the present invention may contain other active ingredients and ingredients for formulation as far as the effect of the present invention is not interfered. Examples of other active ingredients include, for example, hyaluronic acid. The mixing amount of other active ingredients can be appropriately changed depending on the individual effect.

Examples of pharmaceutically acceptable carriers for use in formulating into pharmaceutical preparations include a diluent, a binder (syrup, gum arabic, gelatin, sorbitol, tragacanth, or polyvinyl pyrrolidone), an excipient (lactose, sucrose, corn starch, potassium phosphate, sorbitol, or glycine), a lubricant (magnesium stearate, talc, polyethylene glycol, or silica), a disintegrator (potato starch) and a wetting agent (sodium lauryl sulfate). The present pharmaceutical preparation can be produced by mixing the present peptide, other active ingredients, a pharmaceutically acceptable carrier and so on according to a conventionally known method.

### EXAMPLES

In the following, the present invention will be described more specifically by way of examples, comparative examples, and test examples, however, it is to be noted that the present invention is not limited in any way by these examples.

### Examples 1 to II

The following peptides were synthesized by the peptide solid phase synthesis method as described above.

(Example 1) (Pro-Hyp-Gly)₅ [(POG)₅]

(Example 2) (Pro-Hyp-Gly)₂ [(POG)₂]

(Example 3) Gly-Pro [GP]

(Example 4) Pro-Ala-Gly [PAG]

(Example 5) Pro-Hyp-Gly [POG]

(Example 6) Glu-Hyp [EO]

(Example 7) Ala-Hyp-Gly [AOG]

(Example 8) Glu-Hyp-Gly [EOG]

(Example 9) Hyp-Gly [OG]

(Example 10) Ser-Hyp-Gly [SOG]

(Example 11) Phe-Hyp [FO]

### Example 12

A collagen peptide mixture “Type-S” (available from Nitta Gelatin Inc.).

Analysis by LC-MS/MS revealed that this collagen peptide mixture contained the following peptides.

GP: 672 ppm, PAG: 12520 ppm, POG: 340 ppm, EO: 58 ppm, AOG: 331 ppm, EOG: 308 ppm, OG: 14581 ppm, SOG: 283 ppm, FO: 283 ppm.

### Example 13

A collagen peptide mixture “LCP” (available from Nitta Gelatin Inc.).

Analysis by LC-MS/MS revealed that this collagen peptide mixture contained the following peptides.

GP: 249 ppm, PAG: 15568 ppm, POG: not detected, EO: 14 ppm, AOG: 29 ppm, EOG: 126 ppm, OG: 545 ppm, SOG: 3 ppm, FO: not detected.

### Comparative Examples 1 to 6

The following peptides were synthesized by the peptide solid phase synthesis method as described above.

(Comparative Example 1) Gly-Pro-Hyp [GPO]

(Comparative Example 2) Ala-Hyp [AO]

(Comparative Example 3) Leu-Hyp [LO]

(Comparative Example 4) Pro-Gly [PG]

(Comparative Example 5) Pro-Pro [PP]

(Comparative Example 6) Pro-Ala [PA]

### Comparative Examples 7 to 23

(Comparative Example 7) L-proline (available from Wako Pure Chemical Industries, Ltd.)

(Comparative Example 8) L-hydroxyproline (available from TOKYO CHEMICAL INDUSTRY CO., LTD.)

(Comparative Example 9) L-glycine (available from KANTO CHEMICAL CO., INC.)

(Comparative Example 10) L-leucine (available from KANTO CHEMICAL CO., INC.)

(Comparative Example 11) L-glutamic acid (available from KANTO CHEMICAL CO., INC.)

(Comparative Example 12) L-alanine (available from KANTO CHEMICAL CO., INC.)

(Comparative Example 13) L-serine (available from Wako Pure Chemical Industries, Ltd.)

(Comparative Example 14) L-phenylalanine (available from Wako Pure Chemical Industries, Ltd.)

(Comparative Example 15) L-glycine (available from KANTO CHEMICAL CO., INC.)+hydroxyproline (available from TOKYO CHEMICAL INDUSTRY CO., LTD.)

(Comparative Example 16) L-proline (available from Wako Pure Chemical Industries, Ltd.)+L-alanine (available from KANTO CHEMICAL CO., INC.)+L-glycine (available from KANTO CHEMICAL CO., INC.)

(Comparative Example 17) L-proline (available from Wako Pure Chemical Industries, Ltd.)+L-hydroxyproline (available from TOKYO CHEMICAL INDUSTRY CO., LTD.)+L-glycine (available from KANTO CHEMICAL CO., INC.)

(Comparative Example 18) L-glutamic acid (available from KANTO CHEMICAL CO., INC.)+L-hydroxyproline (available from TOKYO CHEMICAL INDUSTRY CO., LTD.)

(Comparative Example 19) L-alanine (available from KANTO CHEMICAL CO., INC.)+L-hydroxyproline (available from TOKYO CHEMICAL INDUSTRY CO., LTD.)+L-glycine (available from KANTO CHEMICAL CO., INC.)

(Comparative Example 20) L-glutamic acid (available from KANTO CHEMICAL CO., INC.)+L-hydroxyproline (available from TOKYO CHEMICAL INDUSTRY CO., LTD.)+L-glycine (available from KANTO CHEMICAL CO., INC.)

(Comparative Example 21) L-hydroxyproline (available from TOKYO CHEMICAL INDUSTRY CO., LTD.)+L-glycine (available from KANTO CHEMICAL CO., INC.)

(Comparative Example 22) L-serine (available from Wako Pure Chemical Industries, Ltd.)+L-hydroxyproline (available from TOKYO CHEMICAL INDUSTRY CO., LTD.)+L-glycine (available from KANTO CHEMICAL CO., INC.)

(Comparative Example 23) L-phenylalanine (available from Wako Pure Chemical Industries, Ltd.)+L-hydroxyproline (available from TOKYO CHEMICAL INDUSTRY CO., LTD.)

### Comparative Example 24

A collagen peptide mixture derived from jelly fish was prepared according to the method described in NPD 3. Concretely, gelatin was extracted by heating from jelly fish according to an ordinary method, trypsin (available from Sigma) was added in a ratio of 1/100 to the substrate, and the mixture was reacted at 45° C. for 3 hours (pH 7.0). Further, Properase E (available from Genencor KYOWA) was added in a ratio of 1/50 to the substrate, and the mixture was reacted at 50° C. for 3 hours (pH 7.5). The reaction was kept at 95° C. for 5 minutes to inactivate the enzymes. Then, centrifugation (5000 g×15 minutes) was conducted, and the supernatant was collected to obtain a collagen peptide mixture. Analysis by LC-MS/MS revealed that this collagen peptide mixture contained the following peptides.

GP: 5 ppm, PAG: 2 ppm, POG: 7 ppm, EO: not detected, AOG: 2 ppm, EOG: 2 ppm, OG: 10 ppm, SOG: 1 ppm, FO: not detected.

### Comparative Example 25

A collagen peptide mixture “HACP (derived from pig)” (available from JELLICE).

Analysis by LC-MS/MS revealed that this collagen peptide mixture contained the following peptides.

GP: 359 ppm, PAG: 761 ppm, POG: 14 ppm, EO: 271 ppm, AOG: 25 ppm, EOG: not detected, OG: 75 ppm, SOG: not detected, FO: not detected.

### Comparative Example 26

Deerhorn glue was purchased from Siwon Herbal Medicine Co. Analysis by LC-MS/MS revealed that none of the present peptides was detected in deerhorn glue of Comparative Example 26.

### Comparative Example 27

A marine collagen peptide was purchased from IHARA & CO., LTD.

Analysis by LC-MS/MS revealed that the following peptides were contained in the collagen peptide mixture of Comparative Example 27.

GP: not detected, PAG: 58 ppm, POG: 15 ppm, EO: 128 ppm, AOG: 34 ppm, EOG: 104 ppm, OG: 378 ppm, SOG: not detected, FO: not detected.

### Test Example 1

### ET-1 Expression Suppression Test/K10, TGM1, ivl, FLG and HAS2 Expression Promotion Test

A normal human epidermal keratinocyte NHEK (NB) (available from KURABO INDUSTRIES LTD.) was used. The cells were precultured in HuMedia-KG2 (available from KURABO INDUSTRIES LTD.), and 1.5×10⁴ cells/mL×5 mL (7.5×10⁴ cells/dish) were cultured in a 60 mm laboratory dish for two days. After confirming that the cells were subconfluent, the medium was replaced by 5 mL of HuMedia-KB2 (available from KURABO INDUSTRIES LTD.). Samples were added in respective concentrations, and allowed to react for 24 hours (ET-1, K10), 48 hours (TGM1, ivl, HAS2), and 72 hours (FLG). Total RNA was extracted from the cells, reverse transcribed, and subjected to real time PCR. In the real time PCR, as target genes, endoserine-1 (ET-1; Hs00174961_m1), keratin 10 (K10; Hs01043114_g1), transglutaminase 1 (TGM1; Hs01070310_m1), involucrin (ivl; Hs00846307_s1), filaggrin (FLG; Hs00856927_g1) and hyaluronic acid synthase 2 (HAS2; Hs00193435_m1) were measured. GAPDH was used as a correction gene. Calculation was conducted by using a calibration curve method, and FAM pigment of TaqMan Gene Expression was used in primers and probes.

For each peptide or the like of Examples 1 to 13 and Comparative Examples 1 to 27, RNA expression amounts of endoserine-1 (ET-1), keratin 10 (K10), transglutaminase 1 (TGM1), involucrin (ivl), filaggrin (FLG) and hyaluronic acid synthase 2 (HAS2) were measured. The results are shown in Tables 1 to 12. Values in the tables are each average value±standard deviation, and *, ** and *** denote that they are significant with P<0.05, P<0.01, and P<0.001, respectively with respect to the control in Paired-t-test.

TABLE 1

| | | | Endoserine-I |
|---|---|---|---|
| | | Concen- | RNA expression |
| | | tration | amount |
| Control | — | — | 1.00 ± 0.07 |
| Example 1 | (POG)₅ | 0.5 mM | 0.32 ± 0.00* |
| Example 2 | (POG)₂ | 0.5 mM | 0.43 ± 0.03*** |
| Example 3 | GP | 0.5 mM | 0.36 ± 0.12** |
| Example 4 | PAG | 0.5 mM | 0.67 ± 0.01** |
| Example 5 | POG | 0.5 mM | 0.53 ± 0.00 |
| Example 6 | EO | 0.5 mM | 0.65 ± 0.02 |
| Example 7 | AOG | 0.5 mM | 0.52 ± 0.01 |
| Example 8 | EOG | 0.5 mM | 0.71 ± 0.03 |
| Example 9 | OG | 0.5 mM | 0.55 ± 0.09** |
| Example 10 | SOG | 0.5 mM | 0.89 ± 0.07* |
| Example 11 | FO | 0.5 mM | 0.97 ± 0.16 |
| Comparative Example 1 | GPO | 0.5 mM | 1.05 ± 0.11 |
| Comparative Example 2 | AO | 0.5 mM | 1.23 ± 0.03* |
| Comparative Example 3 | LO | 0.5 mM | 1.08 ± 0.00 |
| Comparative Example 4 | PG | 0.5 mM | 1.06 ± 0.10** |
| Comparative Example 5 | PP | 0.5 mM | 1.65 ± 0.07* |
| Comparative Example 6 | PA | 0.5 mM | 1.09 ± 0.01** |
| Comparative Example 7 | P | 0.5 mM | 1.89 ± 0.06** |
| Comparative Example 8 | O | 0.5 mM | 1.52 ± 0.02*** |
| Comparative Example 9 | G | 0.5 mM | 1.01 ± 0.05** |
| Comparative Example 10 | L | 0.5 mM | 1.58 ± 0.05** |
| Comparative Example 11 | E | 0.5 mM | 1.00 ± 0.02** |
| Comparative Example 12 | A | 0.5 mM | 1.58 ± 0.05** |
| Comparative Example 13 | S | 0.5 mM | 1.64 ± 0.07** |
| Comparative Example 14 | F | 0.5 mM | 1.05 ± 0.05 |
| Comparative Example 15 | G + P | 0.5 mM | 1.34 ± 0.03** |
| Comparative Example 16 | P + A + G | 0.5 mM | 1.62 ± 0.07* |
| Comparative Example 17 | P + O + G | 0.5 mM | 1.01 ± 0.07 |
| Comparative Example 18 | E + O | 0.5 mM | 1.35 ± 0.05** |
| Comparative Example 19 | A + O + G | 0.5 mM | 1.95 ± 0.04** |
| Comparative Example 20 | E + O + G | 0.5 mM | 1.34 ± 0.06** |
| Comparative Example 21 | O + G | 0.5 mM | 1.02 ± 0.00** |
| Comparative Example 22 | S + O + G | 0.5 mM | 1.45 ± 0.00** |
| Comparative Example 23 | F + O | 0.5 mM | 1.67 ± 0.05** |

TABLE 2

| | | | Keratin 10 |
|---|---|---|---|
| | | Concen- | RNA expression |
| | | tration | amount |
| Control | — | — | 1.00 ± 0.05 |
| Ca | — | 1.2 mM | 7.45 ± 0.40*** |
| Example 1 | (POG)₅ | 0.5 mM | 6.40 ± 0.06 |
| Example 2 | (POG)₂ | 0.5 mM | 5.69 ± 0.28*** |
| Example 3 | GP | 0.5 mM | 2.56 ± 0.22*** |
| Example 4 | PAG | 0.5 mM | 3.98 ± 0.23*** |
| Example 5 | POG | 0.5 mM | 3.81 ± 0.17*** |
| Example 6 | EO | 0.5 mM | 3.48 ± 0.05*** |
| Example 7 | AOG | 0.5 mM | 5.06 ± 0.37*** |
| Example 8 | EOG | 0.5 mM | 2.39 ± 0.02* |
| Example 9 | OG | 0.5 mM | 1.99 ± 0.13* |
| Example 10 | SOG | 0.5 mM | 1.77 ± 0.04** |
| Example 11 | FO | 0.5 mM | 2.22 ± 0.01*** |
| Comparative Example 1 | GPO | 0.5 mM | 0.94 ± 0.10 |
| Comparative Example 2 | AO | 0.5 mM | 1.00 ± 0.06 |
| Comparative Example 3 | LO | 0.5 mM | 1.04 ± 0.06 |
| Comparative Example 4 | PG | 0.5 mM | 0.89 ± 0.01* |
| Comparative Example 5 | PP | 0.5 mM | 0.98 ± 0.16 |
| Comparative Example 6 | PA | 0.5 mM | 0.70 ± 0.04 |
| Comparative Example 7 | P | 0.5 mM | 0.73 ± 0.03** |
| Comparative Example 8 | O | 0.5 mM | 0.64 ± 0.02** |
| Comparative Example 9 | G | 0.5 mM | 0.81 ± 0.05 |
| Comparative Example 10 | L | 0.5 mM | 0.62 ± 0.01** |
| Comparative Example 11 | E | 0.5 mM | 0.65 ± 0.01** |
| Comparative Example 12 | A | 0.5 mM | 0.82 ± 0.02* |
| Comparative Example 13 | S | 0.5 mM | 0.69 ± 0.03** |
| Comparative Example 14 | F | 0.5 mM | 0.78 ± 0.07 |
| Comparative Example 15 | G + P | 0.5 mM | 0.95 ± 0.08 |
| Comparative Example 16 | P + A + G | 0.5 mM | 0.89 ± 0.43* |
| Comparative Example 17 | P + O + G | 0.5 mM | 0.61 ± 0.02** |
| Comparative Example 18 | E + O | 0.5 mM | 0.95 ± 0.07 |
| Comparative Example 19 | A + O + G | 0.5 mM | 0.68 ± 0.08** |
| Comparative Example 20 | E + O + G | 0.5 mM | 0.99 ± 0.50 |
| Comparative Example 21 | O + G | 0.5 mM | 0.53 ± 0.02** |
| Comparative Example 22 | S + O + G | 0.5 mM | 0.74 ± 0.02** |
| Comparative Example 23 | F + O | 0.5 mM | 0.85 ± 0.04** |

TABLE 3

| | | | Transglutaminase 1 |
|---|---|---|---|
| | | Concen- | RNA expression |
| | | tration | amount |
| Control | — | — | 1.00 ± 0.04 |
| Ca | — | 1.2 mM | 4.21 ± 0.19** |
| Example 1 | (POG)₅ | 0.5 mM | 13.50 ± 2.69** |
| Example 2 | (POG)₂ | 0.5 mM | 7.21 ± 0.03** |
| Example 3 | GP | 0.5 mM | 2.15 ± 0.02** |
| Example 4 | PAG | 0.5 mM | 3.11 ± 0.02*** |
| Example 5 | POG | 0.5 mM | 5.36 ± 0.01*** |
| Example 6 | EO | 0.5 mM | 2.12 ± 0.01*** |
| Example 7 | AOG | 0.5 mM | 2.00 ± 0.02*** |
| Example 8 | EOG | 0.5 mM | 1.69 ± 0.01*** |
| Example 9 | OG | 0.5 mM | 1.99 ± 0.06* |
| Example 10 | SOG | 0.5 mM | 1.21 ± 0.01 |
| Example 11 | FO | 0.5 mM | 1.46 ± 0.02* |
| Comparative Example 1 | GPO | 0.5 mM | 0.98 ± 0.05 |
| Comparative Example 2 | AO | 0.5 mM | 0.48 ± 0.02*** |
| Comparative Example 3 | LO | 0.5 mM | 0.58 ± 0.01*** |
| Comparative Example 4 | PG | 0.5 mM | 0.64 ± 0.01*** |
| Comparative Example 5 | PP | 0.5 mM | 0.64 ± 0.01*** |
| Comparative Example 6 | PA | 0.5 mM | 0.94 ± 0.13 |
| Comparative Example 7 | P | 0.5 mM | 1.02 ± 0.13 |
| Comparative Example 8 | O | 0.5 mM | 0.94 ± 0.10 |
| Comparative Example 9 | G | 0.5 mM | 0.75 ± 0.01** |
| Comparative Example 10 | L | 0.5 mM | 0.63 ± 0.07* |
| Comparative Example 11 | E | 0.5 mM | 0.40 ± 0.01** |
| Comparative Example 12 | A | 0.5 mM | 0.95 ± 0.04 |
| Comparative Example 13 | S | 0.5 mM | 0.85 ± 0.02 |
| Comparative Example 14 | F | 0.5 mM | 0.72 ± 0.05** |
| Comparative Example 15 | G + P | 0.5 mM | 0.60 ± 0.06** |
| Comparative Example 16 | P + A + G | 0.5 mM | 0.38 ± 0.00** |
| Comparative Example 17 | P + O + G | 0.5 mM | 0.69 ± 0.00** |
| Comparative Example 18 | E + O | 0.5 mM | 0.38 ± 0.00** |
| Comparative Example 19 | A + O + G | 0.5 mM | 0.70 ± 0.00** |
| Comparative Example 20 | E + O + G | 0.5 mM | 0.37 ± 0.03** |
| Comparative Example 21 | O + G | 0.5 mM | 0.77 ± 0.03** |
| Comparative Example 22 | S + O + G | 0.5 mM | 0.37 ± 0.02** |
| Comparative Example 23 | F + O | 0.5 mM | 0.68 ± 0.04** |

TABLE 4

| | | | Involucrin |
|---|---|---|---|
| | | Concen- | RNA expression |
| | | tration | amount |
| Control | — | — | 1.00 ± 0.01 |
| Ca | — | 1.2 mM | 0.79 ± 0.01 |
| Example 1 | (POG)₅ | 0.5 mM | 7.96 ± 0.60** |
| Example 2 | (POG)₂ | 0.5 mM | 4.35 ± 0.04*** |
| Example 3 | GP | 0.5 mM | 2.18 ± 0.00*** |
| Example 4 | PAG | 0.5 mM | 2.75 ± 0.03*** |
| Example 5 | POG | 0.5 mM | 1.75 ± 0.22* |
| Example 6 | EO | 0.5 mM | 1.95 ± 0.08** |
| Example 7 | AOG | 0.5 mM | 1.36 ± 0.07* |
| Example 8 | EOG | 0.5 mM | 1.89 ± 0.03* |
| Example 9 | OG | 0.5 mM | 2.04 ± 0.02* |
| Example 10 | SOG | 0.5 mM | 1.59 ± 0.05* |
| Example 11 | FO | 0.5 mM | 1.85 ± 0.00*** |
| Comparative Example 1 | GPO | 0.5 mM | 0.95 ± 0.08 |
| Comparative Example 2 | AO | 0.5 mM | 1.01 ± 0.00 |
| Comparative Example 3 | LO | 0.5 mM | 0.89 ± 0.09* |
| Comparative Example 4 | PG | 0.5 mM | 0.72 ± 0.02* |
| Comparative Example 5 | PP | 0.5 mM | 0.56 ± 0.04* |
| Comparative Example 6 | PA | 0.5 mM | 0.81 ± 0.06** |
| Comparative Example 7 | P | 0.5 mM | 0.78 ± 0.01** |
| Comparative Example 8 | O | 0.5 mM | 0.94 ± 0.04 |
| Comparative Example 9 | G | 0.5 mM | 0.71 ± 0.03** |
| Comparative Example 10 | L | 0.5 mM | 0.84 ± 0.03 |
| Comparative Example 11 | E | 0.5 mM | 0.85 ± 0.11 |
| Comparative Example 12 | A | 0.5 mM | 0.64 ± 0.15** |
| Comparative Example 13 | S | 0.5 mM | 0.98 ± 0.05 |
| Comparative Example 14 | F | 0.5 mM | 0.87 ± 0.04 |
| Comparative Example 15 | G + P | 0.5 mM | 0.68 ± 0.08** |
| Comparative Example 16 | P + A + G | 0.5 mM | 0.77 ± 0.03* |
| Comparative Example 17 | P + O + G | 0.5 mM | 0.96 ± 0.03 |
| Comparative Example 18 | E + O | 0.5 mM | 0.96 ± 0.05 |
| Comparative Example 19 | A + O + G | 0.5 mM | 0.81 ± 0.07 |
| Comparative Example 20 | E + O + G | 0.5 mM | 0.72 ± 0.02 |
| Comparative Example 21 | O + G | 0.5 mM | 0.82 ± 0.01** |
| Comparative Example 22 | S + O + G | 0.5 mM | 0.94 ± 0.05 |
| Comparative Example 23 | F + O | 0.5 mM | 0.76 ± 0.08** |

TABLE 5

| | | | Filaggrin |
|---|---|---|---|
| | | Concen- | RNA expression |
| | | tration | amount |
| Control | — | — | 1.00 ± 0.09 |
| Example 1 | (POG)₅ | 0.5 mM | 11.23 ± 1.56*** |
| Example 2 | (POG)₂ | 0.5 mM | 9.58 ± 1.02*** |
| Example 3 | GP | 0.5 mM | 7.21 ± 0.95*** |
| Example 4 | PAG | 0.5 mM | 5.33 ± 1.25*** |
| Example 5 | POG | 0.5 mM | 5.32 ± 0.69*** |
| Example 6 | EO | 0.5 mM | 3.26 ± 1.61** |
| Example 7 | AOG | 0.5 mM | 3.09 ± 0.23** |
| Example 8 | EOG | 0.5 mM | 2.92 ± 0.89** |
| Example 9 | OG | 0.5 mM | 2.98 ± 0.58** |
| Example 10 | SOG | 0.5 mM | 2.29 ± 0.26** |
| Example 11 | FO | 0.5 mM | 1.95 ± 0.19* |
| Comparative Example 1 | GPO | 0.5 mM | 1.08 ± 0.21 |
| Comparative Example 2 | AO | 0.5 mM | 0.98 ± 0.95 |
| Comparative Example 3 | LO | 0.5 mM | 0.69 ± 0.58 |
| Comparative Example 4 | PG | 0.5 mM | 0.86 ± 0.18 |
| Comparative Example 5 | PP | 0.5 mM | 0.66 ± 0.09** |
| Comparative Example 6 | PA | 0.5 mM | 0.63 ± 0.08** |
| Comparative Example 7 | P | 0.5 mM | 0.85 ± 0.18 |
| Comparative Example 8 | O | 0.5 mM | 0.89 ± 0.09 |
| Comparative Example 9 | G | 0.5 mM | 0.87 ± 0.05 |
| Comparative Example 10 | L | 0.5 mM | 0.89 ± 0.08 |
| Comparative Example 11 | E | 0.5 mM | 0.77 ± 0.04* |
| Comparative Example 12 | A | 0.5 mM | 0.69 ± 0.02** |
| Comparative Example 13 | S | 0.5 mM | 0.85 ± 0.08 |
| Comparative Example 14 | F | 0.5 mM | 0.59 ± 0.05** |
| Comparative Example 15 | G + P | 0.5 mM | 0.99 ± 0.08 |
| Comparative Example 16 | P + A + G | 0.5 mM | 0.69 ± 0.04** |
| Comparative Example 17 | P + O + G | 0.5 mM | 0.79 ± 0.07* |
| Comparative Example 18 | E + O | 0.5 mM | 0.80 ± 0.04 |
| Comparative Example 19 | A + O + G | 0.5 mM | 1.00 ± 0.12 |
| Comparative Example 20 | E + O + G | 0.5 mM | 0.98 ± 0.02 |
| Comparative Example 21 | O + G | 0.5 mM | 0.85 ± 0.08 |
| Comparative Example 22 | S + O + G | 0.5 mM | 1.04 ± 0.09 |
| Comparative Example 23 | F + O | 0.5 mM | 0.95 ± 0.08 |

TABLE 6

| | | | Hyaluronic acid |
|---|---|---|---|
| | | | synthase 2 |
| | | Concen- | RNA expression |
| | | tration | amount |
| Control | — | — | 1.00 ± 0.09 |
| Example 1 | (POG)₅ | 0.5 mM | 3.45 ± 1.02** |
| Example 2 | (POG)₂ | 0.5 mM | 3.11 ± 0.89** |
| Example 3 | GP | 0.5 mM | 3.08 ± 0.99** |
| Example 4 | PAG | 0.5 mM | 2.99 ± 1.03** |
| Example 5 | POG | 0.5 mM | 2.45 ± 0.90** |
| Example 6 | EO | 0.5 mM | 2.35 ± 0.85** |
| Example 7 | AOG | 0.5 mM | 2.21 ± 0.67** |
| Example 8 | EOG | 0.5 mM | 2.56 ± 0.45** |
| Example 9 | OG | 0.5 mM | 2.18 ± 0.67** |
| Example 10 | SOG | 0.5 mM | 2.14 ± 0.89* |
| Example 11 | FO | 0.5 mM | 1.63 ± 0.49* |
| Comparative Example 1 | GPO | 0.5 mM | 0.98 ± 0.45 |
| Comparative Example 2 | AO | 0.5 mM | 0.99 ± 0.66 |
| Comparative Example 3 | LO | 0.5 mM | 0.87 ± 0.63 |
| Comparative Example 4 | PG | 0.5 mM | 0.54 ± 0.98* |
| Comparative Example 5 | PP | 0.5 mM | 0.78 ± 0.64* |
| Comparative Example 6 | PA | 0.5 mM | 0.99 ± 0.68 |
| Comparative Example 7 | P | 0.5 mM | 0.84 ± 0.47 |
| Comparative Example 8 | O | 0.5 mM | 0.70 ± 0.29 |
| Comparative Example 9 | G | 0.5 mM | 0.61 ± 0.40** |
| Comparative Example 10 | L | 0.5 mM | 0.76 ± 0.07* |
| Comparative Example 11 | E | 0.5 mM | 1.06 ± 0.24 |
| Comparative Example 12 | A | 0.5 mM | 0.89 ± 0.57 |
| Comparative Example 13 | S | 0.5 mM | 0.81 ± 0.50 |
| Comparative Example 14 | F | 0.5 mM | 0.89 ± 0.84 |
| Comparative Example 15 | G + P | 0.5 mM | 1.02 ± 0.19 |
| Comparative Example 16 | P + A + G | 0.5 mM | 0.78 ± 0.49 |
| Comparative Example 17 | P + O + G | 0.5 mM | 0.59 ± 0.07* |
| Comparative Example 18 | E + O | 0.5 mM | 0.87 ± 0.37 |
| Comparative Example 19 | A + O + G | 0.5 mM | 1.09 ± 0.28 |
| Comparative Example 20 | E + O + G | 0.5 mM | 0.99 ± 0.64 |
| Comparative Example 21 | O + G | 0.5 mM | 0.87 ± 0.37 |
| Comparative Example 22 | S + O + G | 0.5 mM | 1.08 ± 0.13 |
| Comparative Example 23 | F + O | 0.5 mM | 0.82 ± 0.19 |

TABLE 7

| | | Endoserine-I |
|---|---|---|
| | Concentration | RNA expression amount |
| Control | — | 1.00 ± 0.05 |
| Example 12 | 0.10% | 0.32 ± 0.04** |
| Example 13 | 0.10% | 0.76 ± 0.03** |
| Comparative Example 24 | 0.10% | 1.20 ± 0.01** |
| Comparative Example 25 | 0.10% | 1.15 ± 0.02* |
| Comparative Example 26 | 0.10% | 1.04 ± 0.06 |
| Comparative Example 27 | 0.10% | 1.25 ± 0.04 |

TABLE 8

| | | Keratin 10 |
|---|---|---|
| | Concentration | RNA expression amount |
| Control | — | 1.00 ± 0.00 |
| Example 12 | 0.10% | 2.35 ± 0.02** |
| Example 13 | 0.10% | 1.51 ± 0.03** |
| Comparative Example 24 | 0.10% | 1.02 ± 0.01 |
| Comparative Example 25 | 0.10% | 0.95 ± 0.03 |
| Comparative Example 26 | 0.10% | 0.99 ± 0.08 |
| Comparative Example 27 | 0.10% | 0.82 ± 0.05 |

TABLE 9

| | | Transglutaminase 1 |
|---|---|---|
| | Concentration | RNA expression amount |
| Control | — | 1.00 ± 0.02 |
| Example 12 | 0.10% | 3.26 ± 0.09** |
| Example 13 | 0.10% | 1.69 ± 0.02** |
| Comparative Example 24 | 0.10% | 0.76 ± 0.01** |
| Comparative Example 25 | 0.10% | 0.88 ± 0.03* |
| Comparative Example 26 | 0.10% | 1.04 ± 0.05 |
| Comparative Example 27 | 0.10% | 1.09 ± 0.03 |

TABLE 10

| | | Involucrin |
|---|---|---|
| | Concentration | RNA expression amount |
| Control | — | 1.00 ± 0.01 |
| Example 12 | 0.10% | 2.92 ± 0.04** |
| Example 13 | 0.10% | 1.80 ± 0.01** |
| Comparative Example 24 | 0.10% | 0.95 ± 0.00* |
| Comparative Example 25 | 0.10% | 0.97 ± 0.02 |
| Comparative Example 26 | 0.10% | 1.00 ± 0.08 |
| Comparative Example 27 | 0.10% | 0.92 ± 0.10 |

TABLE 11

| | | Filaggrin |
|---|---|---|
| | Concentration | RNA expression amount |
| Control | — | 1.00 ± 0.07 |
| Example 12 | 0.10% | 3.58 ± 0.15*** |
| Example 13 | 0.10% | 2.09 ± 0.09* |
| Comparative Example 24 | 0.10% | 1.01 ± 0.08 |
| Comparative Example 25 | 0.10% | 0.98 ± 0.10 |
| Comparative Example 26 | 0.10% | 1.12 ± 0.11 |
| Comparative Example 27 | 0.10% | 0.99 ± 0.07 |

TABLE 12

| | | Hyaluronic acid synthase 2 |
|---|---|---|
| | Concentration | RNA expression amount |
| Control | — | 1.00 ± 0.11 |
| Example 12 | 0.10% | 4.02 ± 0.45** |
| Example 13 | 0.10% | 3.48 ± 0.13** |
| Comparative Example 24 | 0.10% | 1.08 ± 0.04 |
| Comparative Example 25 | 0.10% | 0.87 ± 0.19 |
| Comparative Example 26 | 0.10% | 1.00 ± 0.19 |
| Comparative Example 27 | 0.10% | 1.10 ± 0.08 |

### Test Example 2

### Test with Dry Skin Model Mouse

Male hairless mice aged at 5 weeks (HOS: HR-1) were divided into three groups, and a group fed with a normal feed (N group), a control group fed with a special feed for inducing dry skin (HR-AD, available from Nosan Corporation) (C group), and a group fed with a feed prepared by mixing 1% by weight of a collagen peptide into HR-AD (CP group) were provided. After 5 weeks from the start of administration of the feed, the water content of the skin of the back was measured with a Corneometer. The results are shown in Table 13. At the same time, transepidermal water loss (TWEL) was measured with a Tewameter. The results are shown in Table 14. Values in the table are each average value±standard deviation, and * and ** denote that they are significant with P<0.05 and P<0.01, respectively with respect to the control group (C group) in Paired-t-test.

TABLE 13

| | Corneometer score |
|---|---|
| N group | 57.69 ± 5.63** |
| C group | 46.92 ± 3.69 |
| CP group (Example 12) | 56.99 ± 5.22** |
| CP group (Example 13) | 53.85 ± 4.95** |
| CP group (Comparative Example 24) | 46.62 ± 3.99 |
| CP group (Comparative Example 25) | 47.35 ± 3.67 |
| CP group (Comparative Example 26) | 46.98 ± 4.28 |
| CP group (Comparative Example 27) | 49.88 ± 4.33 |

TABLE 14

| | TWEL (g/hr/m²) |
|---|---|
| N group | 4.60 ± 0.23 |
| C group | 21.72 ± 1.89 |
| CP group (Example 12) | 7.51 ± 0.34** |
| CP group (Example 13) | 10.13 ± 1.12* |
| CP group (Comparative Example 24) | 20.90 ± 1.75 |
| CP group (Comparative Example 25) | 17.33 ± 1.99 |
| CP group (Comparative Example 26) | 20.99 ± 1.77 |
| CP group (Comparative Example 27) | 18.92 ± 1.56 |

### Test Example 3

### Measurement of Blood IgE Concentration in Ovalbumin (OVA) Sensitized Mouse

BALB/c male mice aged at 6 weeks were sensitized by intraperitoneally administering 20 μg/mouse of OVA. After sensitization, the mice were divided into seven groups, and a control sample, and a test sample prepared by mixing 1% by weight of a collagen peptide of Example 12, 13, Comparative Example 24, 25, 26 or 27 respectively in a control sample were administered. The amount of blood IgE was detected by ELISA kit (E99-115 available from Funakoshi Co., Ltd.). The procedure followed the protocol. The results are shown in Table 15. Values in the table are each average value±standard deviation, and * and ** denote that they are significant with P<0.05 and P<0.01, with respect to the control in Paired-t-test.

TABLE 15

| | IgE value (ng/mL) |
|---|---|
| Control | 7.51 ± 0.61 |
| Example 12 | 3.22 ± 0.19** |
| Example 13 | 4.31 ± 0.44** |
| Comparative Example 24 | 7.34 ± 0.43 |
| Comparative Example 25 | 7.58 ± 0.83 |
| Comparative Example 26 | 7.49 ± 0.48 |
| Comparative Example 27 | 6.99 ± 0.40 |

As can be seen from Table 1, the present peptide suppressed expression of endoserine-1. Accordingly, generation of melanin pigments from melanocytes is suppressed, and pigmentation can be suppressed. As can be seen from Tables 2 to 5, these peptides promoted expression of transglutaminase 1, involucrin, keratin 10, filaggrin and hyaluronic acid synthase 2. Accordingly, it is possible to promote metabolism of epidermis and promote turnover of the skin, and to make the melanin pigments (maculae) in the skin to be discharged more quickly. Further, by promoting expression of transglutaminase 1, involucrin, keratin 10, filaggrin and hyaluronic acid synthase 2, the skin barrier function and the moisture retention are improved, and by reduction of the blood IgE amount, atopic dermatitis can be ameliorated.

As can be seen from Tables 7 to 15, in the collagen peptide mixture derived from jelly fish described in NPD 3 (Comparative Example 24), the collagen peptide mixture comprising only 0.15% by weight of the present peptide (Comparative Example 25), the deerhorn glue described in NPD 4 (Comparative Example 26) and the collagen peptide comprising only 0.07% by weight of the present peptide described in NPD 5 (Comparative Example 27), the effect of suppressing expression of endoserine-1, and the effect of promoting expression of transglutaminase 1, involucrin, keratin 10, filaggrin and hyaluronic acid synthase 2 were not exhibited. In contrast, in the collagen peptide mixture “LCP” comprising 1.65% by weight of the present peptide (available from Nitta Gelatin Inc.: Example 13), these effects were reliably exhibited, and in the collagen peptide mixture “Type-S” comprising 2.94% by weight of the present peptide (available from Nitta Gelatin Inc.: Example 12), both the effect of suppressing expression of endoserine-1, and the effect of promoting expression of transglutaminase 1, involucrin, keratin 10, filaggrin and hyaluronic acid synthase 2 were significantly exhibited.

As can be seen from Table 14, in “LCP” in Example 13 and “Type-S” in Example 12, the TWEL value was improved to a normal value. Similarly, Table 15 reveals that both in Example 12 and Example 13, the blood IgE value is significantly reduced, and an allergic reaction is suppressed. These demonstrate that a collagen peptide comprising 1.6% by weight or more of a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp ameliorates atopic dermatitis by improving the skin barrier function and reducing an allergic reaction.

In conclusion, the present peptide or the like is useful as a whitening promoting agent or atopic dermatitis ameliorating agent.

### INDUSTRIAL APPLICABILITY

The present invention can provide an excellent whitening promoting agent or atopic dermatitis ameliorating agent comprising a peptide or the like.

1. A whitening promoting agent or atopic dermatitis ameliorating agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.

2. The whitening promoting agent or atopic dermatitis ameliorating agent according to claim 1, comprising a collagen peptide mixture comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof so that the total content of the peptide or the pharmaceutically acceptable salt thereof is 1.6% by weight or more.

3. The whitening promoting agent or atopic dermatitis ameliorating agent according claim 1, comprising a collagen peptide mixture comprising a peptide selected from the group consisting of Hyp-Gly, Pro-Ala-Gly, Gly-Pro, Glu-Hyp-Gly, Glu-Hyp, Ala-Hyp-Gly, Ser-Hyp-Gly and Phe-Hyp or a pharmaceutically acceptable salt thereof so that the total content of the peptide or the pharmaceutically acceptable salt thereof is 1.6% by weight or more.

4. The whitening promoting agent or atopic dermatitis ameliorating agent according to claim 1, which is administered orally or percutaneously.

5. An endoserine-1 expression suppressing agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.

6. A filaggrin expression promoting agent comprising a peptide selected from the group consisting of Hyp-Gly, Pro-Ala-Gly, Gly-Pro, Glu-Hyp-Gly, (Pro-Hyp-Gly)₅, (Pro-Hyp-Gly)₂, Pro-Hyp-Gly, Glu-Hyp, Ala-Hyp-Gly, Ser-Hyp-Gly and Phe-Hyp or a pharmaceutically acceptable salt thereof.


SEQUENCE LISTING (2 sequences)

SEQ ID NO: 1 - protein, 15 aa, Sus scrofa
  MISC_FEATURE (2)..(2): 4Hyp
PXGPXGPXGPXGPXG

SEQ ID NO: 2 - protein, 6 aa, Sus scrofa
  MISC_FEATURE (2)..(2): 4Hyp
PXGPXG